(19) United States
(12) Patent Application Publication
(10) Pub. No.: US 2024/0407833 A1
(43) Pub. Date: Dec. 12, 2024

(54) METHOD AND DEVICE FOR FRACTIONAL FAT TREATMENT

(71) Applicant: Inmode Ltd., Yokneam (IL)

(72) Inventor: Michael Kreindel, Richmond Hill (CA)

(73) Assignee: Inmode Ltd., Yokneam (IL)

(21) Appl. No.: 18/329,656

(22) Filed: Jun. 6, 2023

(51) Int. Cl. A61B 18/14

(52) U.S. Cl. CPC A61B 18/148; A61B 2018/00464; A61B 2018/00589; A61B 2018/00761; A61B 2018/00791

(57) ABSTRACT

A method for thermal fat destruction and tightening includes delivering RF energy into adipose tissue under a skin surface to create thermal damage of fat, to cause an alteration of the overlying soft tissue contour and to heat connective tissue to cause collagen contraction. The method includes cannula movement and pulsed RF energy delivery to create a matrix of isolated coagulation zones inside the tissue.

### FIELD OF THE INVENTION

The present invention relates to methods and device for tightening and fat destruction using pulsed RF energy delivered through a minimally-invasive cannula to create isolated coagulation zones in subcutaneous fat.

### BACKGROUND OF THE INVENTION

Fractional injuries to the skin and sub-dermal tissue can be delivered by laser systems such as FRAXEL™, which sends small beams of erbium glass laser beams into the dermis or alternatively fractional devices as micro-needeling, surface ablation or invasive needling. The advantage of these segmental, fractional injury, is the tissue is stimulated with an matrix of micro-traumas providing fractional skin resurfacing, skin tightening, acne scar and wrinkle treatment as well as treatment of hyperhidrosis, acne and trans dermal drug delivery.

U.S. Pat. No. 6,210,402 describes a method for dermatological treatment of an external body surface at applying high frequency electrical energy to the electrode terminal comprising multiple conductive elements.

U.S. Pat. Nos. 8,496,654 and 8,357,157 describe devices for cosmetic fractional epidermis ablation where multiple electrodes applied to the skin surface and RF energy are applied between the multiple electrodes and a grounded return electrode, wherein the plurality of RF application elements are free of any ground electrode therebetween.

U.S. Pat. No. 8,579,896 describes fractional coagulation of skin with one electrode constructed from spaced a part elements.

U.S. Pat. No. 9,108,036 describes a skin treatment device, comprising: plurality of electrodes arranged in a cluster; and a plurality of electrodes sized substantially larger than the first size and arranged at a periphery of the cluster and spaced from the cluster, and wherein the cluster of elements are free of any portion of the larger sized electrode therebetween.

U.S. Pat. No. 9,480,836 describes a needle array penetrating into the skin and powered by a motor connected to the array, wherein RF energy is applied between the needles which penetrating into the skin.

U.S. Pat. No. 9,095,357 provides improved methods and apparatus for skin treatment and tissue remodeling. The apparatus includes an array of needles that penetrate the skin and serve as electrodes to deliver radio frequency (RF) current or other electrical or optical energy into the tissue being treated, causing thermal damage in controlled patterns. The damaged regions promote beneficial results, such as uniform skin tightening by stimulation of wound healing and collagen growth.

An alternative method uses a cannula inserted in sub-cutaneous fat, which delivers thermal energy resulting in fat destruction and connective tissue contraction.

U.S. Pat. No. 6,206,873 describes a method for using laser energy delivered to the subcutaneous through the cannula with a laser fiber. Optical energy heats tissue near the cannula end to destroy fat layer and tighten the tissue.

U.S. Pat. No. 8,103,355 describes use of an RF cannula for fat destruction and tightening of connective tissue and skin.

All these minimally invasive methods describe CW or quasi-CW energy delivery to coagulate a whole layer of fat. This large volume heating may result in seroma and skin burn, so that it requires good skills and techniques and a long learning curve to prevent such unwanted phenomena.

### SUMMARY OF THE INVENTION

The present invention seeks to provide a method and device for adipose tissue destruction and simultaneous tightening while minimizing the mechanical damage of the skin and surrounding subcutaneous tissue. The device is based on a minimally invasive procedure in which a cannula is inserted directly into the adipose tissue and radio-frequency (RF) energy is applied to the cannula tip. The size of the cannula is designed to create higher energy density in the vicinity of the cannula tip. The RF energy density is high enough to create damage to the adipose tissue, which results in coagulation of adipose tissue and tightening fiber septa. The RF energy is delivered in a pulsed manner to create isolated coagulation zones during cannula movement.

The invention creates a matrix of small coagulation zones in a given treatment volume, which achieves the same effect as a large treatment volume but without the risk. The hand piece is moved to deliver energy in a pulse mode and to create a matrix of small coagulation zones in fat tissue.

Isolated coagulation zones are relatively small to preserve a smooth tissue surface appearance. The size of the coagulation zones may be larger for deeper treatment and may be smaller for superficial treatment. The size of the coagulation zones can vary from 1 mm³ to 2 cm³.

The RF energy can be delivered through an electrode located at the distal end of the cannula while a large return electrode is placed on the skin surface. This monopolar configuration allows creating very small coagulation zones. There may be a strong variation in the amount of energy near the active electrode.

In other embodiment, the RF energy can be applied between two or more electrodes located on cannula. In this bipolar configuration, energy is utilized more efficiently and localized between the electrodes. The distance between electrodes may be in the range of 0.5 mm to 10 mm.

The cannula may have electrically insulated tip to avoid skin damage if it is accidentally touched from inside by the cannula.

The treatment cannula may have electrodes only on the side directed inside the fat, while the part directed towards the skin is not conductive. This reduces a risk of accidental skin damage if the cannula is too close to the skin surface.

In an alternative design, a temperature sensor is embedded in the cannula to monitor tissue temperature. The device may adjust RF parameters according to data from the temperature sensor. The following RF parameters, without limitation, can be adjusted: RF power; RF pulse duration; delay between RF pulses; and other correlated parameters such as RF current, RF voltage, energy per pulse.

Preferably, the cannula is isolated to prevent RF energy leak close to the insertion portion. The handle of the cannula may be ergonomic and may have indexing showing orientation of electrodes inside the tissue.

The part of the electrodes coming in contact with the tissue may be made from biocompatible materials. For example, the internal electrode tip can be made from stainless steel or titanium. RF electrodes may have a thin dielectric coating providing capacitive electrical coupling.

The cannula diameter can be in the range of 0.5-5 mm. A larger diameter can be used for a longer cannula, for use in treating larger body areas, while smaller cannulas can be used for treatment of smaller zones on the face or neck.

A movement or position sensor can be disposed in the cannula or handle for controlling the electrode movement. The RF pulse can be released according to feedback from motion sensor. The RF parameters can be adjusted according to the movement speed. Cannula movement should be slow enough to avoid significant displacement during RF pulse duration. For a movement speed of 25 mm per second and an electrode size of 3 mm, the maximal RF pulse width can be calculated as
3 mm/25 [mm/sec]=120 msec

The shorter the pulse, the more RF energy is localized during the cannula movement. The delay between pulses may be at least 120 ms to avoid overlapping between coagulation zones.

The parameters of the RF energy may be adjusted for destruction of adipose tissue and skin tightening. Frequency of RF current may be varied from 200 KHz up to 40 MHz. RF energy can be controlled by controlling the RF power. The other option to control average RF power is delivering constant RF power with a train of pulses and controlling the duty cycle of RF pulses.

The method of the invention may be used, for example, to achieve a reduction in body weight, body shape remodeling, cellulite reduction, loose skin reduction, wrinkle treatment, body surface tightening, skin tightening, and collagen remodeling, among others.

Other types of energy can be used including optical energy generated by plasma, laser, lamp or light emitting diode. Acoustic energy such as ultrasound can also be utilized to perform a similar thermal effect.

The temperature required for the collagen remodeling depends on the heating time. For millisecond range pulses, the temperature may be 50-70° C. If treatment time is a few minutes, the temperature may be 45-50° C. to cause collagen remodeling without skin damage.

In another embodiment, the device may have circuit for measuring tissue impedance. Change of measured impedance between electrodes may provide information about tissue heating and coagulation. Electronic circuits may measure RF current, voltage, impedance or other parameters.

Cooling of the skin beyond the treatment area can be used to avoid damage of skin.

The system for powering and controlling RF energy delivery may include a power supply that converts AC voltage from the wall plug to stabilize DC voltage. An RF generator connected to the power supply may be used to generate high frequency voltage. The RF generator may be designed to maintain constant power in the working range of parameters. The system may have a controller that controls the RF parameters and an user interface, including an LCD screen and a touch screen. The controller may have a microprocessor and dedicated software. The monitoring system may measure RF parameters including tissue impedance and/or RF current and/or RF voltage or other electronic parameters. The system may have a connector to connect one or more electrodes to the system unit.

Thus, in its first aspect, the invention provides a method of thermal fat destruction and collagenous tissue contraction comprising, for each of one or more regions of body: inserting a cannula with at least one RF electrode inside the adipose tissue; delivering RF energy pulses between the cannula electrode and a return electrode applied to the skin surface, wherein the RF energy pulse is sufficient to coagulate a cluster of adipose tissue in a vicinity of the cannula electrode; and moving the cannula inside the subcutaneous tissue essentially parallel to the skin surface (“parallel” encompasses parallel within ±30°). Cannula is inserted into the subdermal fat and moved back and forth similar to liposuction procedure along the skin surface. Cannula movement may be slow enough to avoid significant displacement during RF pulse duration.

In another aspect, the invention provides a method of thermal fat destruction and tissue tightening comprising, for each of one or more regions of the body: inserting a cannula with two or more RF electrodes inside the adipose tissue; and delivering RF energy pulses between the two or more electrodes located on the distal cannula end, wherein the RF energy pulse is sufficient to coagulate a cluster of adipose tissue in vicinity of the cannula electrodes; and moving the cannula inside the tissue. Cannula movement may be slow enough to avoid significant displacement during RF pulse duration.

### BRIEF DESCRIPTION OF THE DRAWINGS

The present invention will be understood and appreciated more fully from the following detailed description taken in conjunction with the drawings in which:

FIG. 1 illustrates a bipolar cannula with two electrodes and RF current applied between the electrodes;

FIG. 2 illustrates the bipolar cannula with two electrodes and isolated tip;

FIG. 3 illustrates a monopolar cannula with RF current around the electrode;

FIG. 4 illustrates a bipolar cannula with two electrodes oriented inside the fat layer.

FIG. 5 illustrates a cannula inserted into the tissue and coagulation zones created by the RF pulses.

### DETAILED DESCRIPTION OF EMBODIMENTS

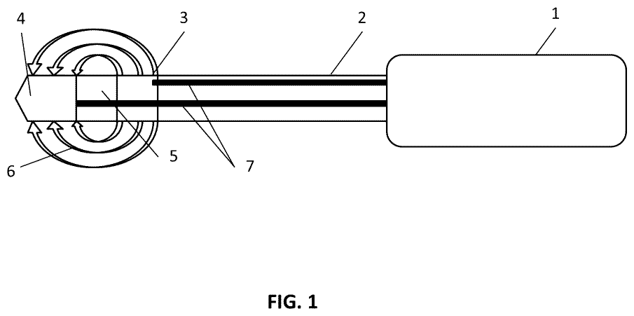

Referring first to FIG. 1, a cannula assembly is shown for applying RF energy inside the adipose tissue in accordance with one embodiment of the invention. The cannula assembly is configured to be connected to an RF generator (not shown). The cannula assembly is configured to be inserted into the human body. The cannula includes shaft 2 having a dielectric surface. Diameter of the shaft can be in the range of 0.5-5 mm, without limitation. Shaft is connected to the handle 1. At the distal end of the shaft 2 are conductive electrodes 3 and 4 connected electrically with RF generator through conductors 7. Electrodes 3 and 4 have opposite polarity and RF current 6 is applied between the electrodes. In this bipolar configuration of cannula tip, the heat is generated mostly in the gap between electrodes 5 and near the surface of the electrodes 3 and 4. The electrode 4 may have a shape for better penetration into the tissue. Handle 1 is designed for convenient use of cannula by operator.

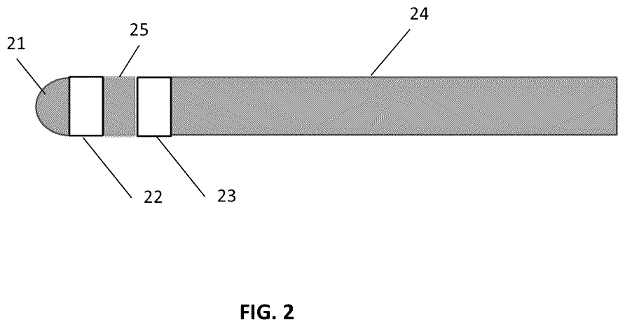

FIG. 2 shows a distal end of the bipolar cannula 24 with two electrodes 22 and 23 spaced with gap 25. The gap 25 is filled with a ring made from dielectric material. The cannula has a non-conductive tip 21 having a non-conductive electrical surface to prevent skin burn due to accidental contact between the cannula tip and the inner side of the skin. The non-conductive tip may have a blunt or sharp end for easier movement inside the tissue.

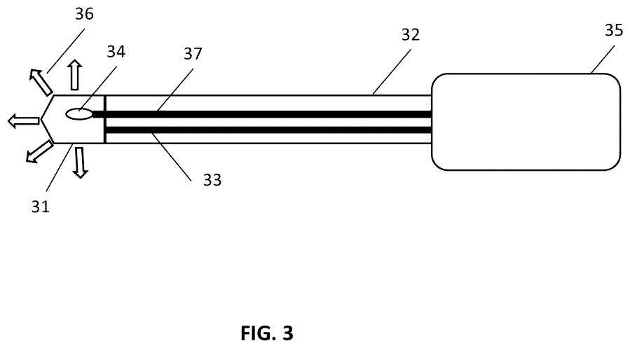

Referring first to FIG. 3, a cannula assembly is shown for applying RF energy inside the adipose tissue in accordance with alternative embodiment of the invention. The electrode 31 is configured to be connected to an RF generator (not shown). The cannula assembly is configured to be inserted into the human body. The cannula includes shaft 32 having a dielectric surface. The diameter of the shaft 32 can be in the range of 0.5-5 mm. Shaft 32 is connected to the handle 35. At the distal end of the shaft 32 are one or more conductive electrodes 31 connected electrically with RF generator through conductors 33. RF current is applied between electrodes 31 and a large return electrode applied to the skin surface and having opposite polarity. In this mono-polar configuration of the device, RF current 36 flows from cannula electrode 31 toward the return electrode. In this mono-polar configuration of cannula, the heat is generated mostly near the surface of the electrodes 31. The electrode 31 may have a shape for better penetration into the tissue. Handle 35 is designed for convenient use of cannula by operator. Temperature sensor 34 is disposed in the cannula near the electrode to monitor tissue heating. The sensor is connected to the controller through wire 37. The controller of the device may adjust RF parameters according to temperature feedback to provide treatment efficiency and improve safety.

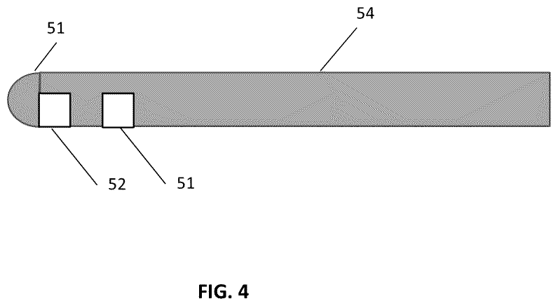

FIG. 4 shows the cannula distal end with electrodes 51 and 52 located on one side of the cannula. This design allows for safe work near the skin surface.

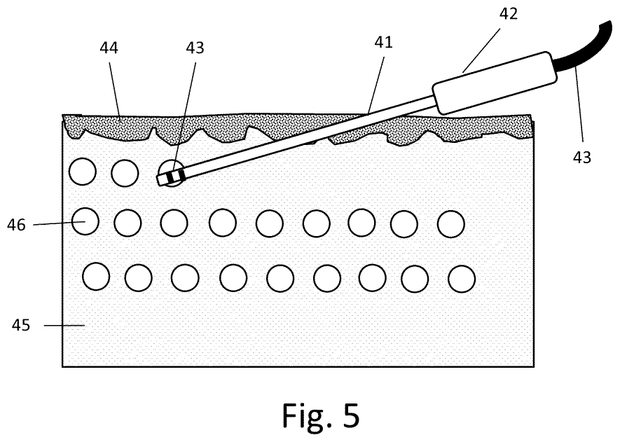

FIG. 5 shows skin layer 44 with an underlying fat layer 45. The shaft of cannula is inserted into the tissue through the skin layer 44 so that the cannula shaft reaches fat layer 45. The cannula is moved inside the fat layer 45 while RF pulses are delivered to the cannula electrodes 43 to create coagulation zones 46. The movement of the cannula is coordinated with RF pulses to create isolated coagulation zones 43. The shaft 41 of the cannula is connected to the handle 42 which is designed to move the cannula. The cannula is connected to the platform with cable 43.

Using the method and device of the invention to treat subcutaneous adipose tissue and tight the skin, the following exemplary parameter values of RF energy may be used: RF frequency: 0.2-40 MHz; pulsed RF power 10 W to 1000 W; RF energy may be delivered in a pulsed manner with a pulse duration of 5-500 ms; delivered energy may create a high enough temperature in the vicinity of electrode tip 12 to destroy adipose tissue. The temperature may exceed the damage threshold of adipose tissue and reach 50° C. or higher.

What is claimed is:

1. A method for thermal fat destruction and tissue tightening comprising:
a) inserting a distal portion of a cannula that has one or more electrodes at the distal portion into adipose tissue;
b) moving the cannula parallel to a skin surface;
c) delivering RF pulses to heat the adipose tissue and create coagulation zones near said distal portion of said cannula; and
d) synchronizing cannula movement and RF pulse delivery to avoid overlapping coagulation zones created by said RF pulses.

2. The method according to claim 1, wherein said RF pulses are applied between two or more electrodes located at the distal portion of the cannula.

3. The method according to claim 1, wherein said RF pulses are applied between said electrode located at said distal portion of the cannula and a return electrode applied to the skin surface.

4. The method according to claim 1, wherein tissue heating is monitored using a thermal sensor.

5. A method for thermal fat destruction and tissue tightening comprising:
a) inserting a distal portion of a cannula with one or more electrodes into adipose tissue;
b) moving said cannula parallel to a skin surface; and
c) delivering RF pulses with a duration shorter than a time required for full displacement of a cannula tip of said cannula, wherein RF pulse amplitude is high enough to coagulate tissue in a vicinity of said cannula tip.

6. The method according to claim 5, wherein cannula movement speed is in a range of 1-10 cm/sec.

7. The method according to claim 5, wherein a pulse duration is in a range of 5-500 ms.

8. The method according to claim 5, wherein RF pulses are applied between two or more electrodes located at the distal portion of the cannula.

9. The method according to claim 5, wherein RF pulses are applied between one or more electrodes located at the distal portion of the cannula and a return electrode applied to the skin surface.

10. The method according to claim 5, wherein said cannula comprises a movement sensor for monitoring cannula movement.

11. The method according to claim 5, wherein said cannula comprises a thermal sensor to monitor tissue heating.

12. The method according to claim 1, wherein an isolated coagulation zone is in a range of 1mm³ to 2cm³.

13. The method according to claim 5, wherein an isolated coagulation zone is in a range of 1mm³ to 2cm³.

14. The method according to claim 1, wherein the method is used to perform:
a) reducing body weight;
b) localized fat reduction
c) cellulite reduction;
d) loose skin reduction;
e) wrinkle treatment;
f) body surface tightening;
g) skin tightening; or
h) collagen remodeling.

15. The method according to claim 5, wherein the method is used to perform:
a) reducing body weight;
b) localized fat reduction
c) cellulite reduction;
d) loose skin reduction;
e) wrinkle treatment;
f) body surface tightening;
g) skin tightening; or
h) collagen remodeling.

16. A method for thermal fat destruction and tissue tightening comprising:
a) inserting a distal portion of a cannula with an energy source into adipose tissue;
b) moving said cannula parallel to a skin surface;
c) delivering energy pulses to heat the adipose tissue and create coagulation zones in a vicinity of the distal portion of the cannula; and
d) synchronizing cannula movement and RF pulse delivery to avoid overlapping coagulation zones created by RF pulses.

17. The method according to claim 16, wherein said energy source comprises any one of the following: electrical current, laser or other optical energy, plasma, ultrasound.
